(19) United States
(12) Patent Application Publication
(10) Pub. No.: US 2012/0041401 A1
(43) Pub. Date: Feb. 16, 2012

(54) MINUS-PRESSURED BANDAGE

(76) Inventors: Aldous CHAO, Nantou County (TW); Wei-Hua Lu, Pingtung County (TW); Yung-Chuan Chen, Kaohsiung City (TW); Li-Wen Chen, Taipei City (TW); Chien-Hsiung Tsai, Kaohsiung City (TW); Li-Chiang Lin, Kaohsiung City (TW); Cheng-Loong Liang, Kaohsiung City (TW); Wei-Te Chen, Tainan County (TW); Wei-Jen Shih, Kaohsiung County (TW)

(21) Appl. No.: 12/956,763

(22) Filed: Nov. 30, 2010

(30) Foreign Application Priority Data
Aug. 13, 2010 (TW) 99127087

(51) Int. Cl. A61M 1/00

(52) U.S. Cl. 604/319

(57) ABSTRACT

A minus-pressured bandage includes a coverage layer, a minus-pressed acting member, an adhering layer and a fixing tap. The coverage layer includes a through-hole to be connected to a minus-pressured device. The minus-pressed acting member includes a fixed-shaped layer and a contact layer abutting with the fixed-shaped layer. The contact layer is made of a resilient material. The fixed-shaped layer has hardness greater than that of the contact layer. Each of the fixed-shaped layer and contact layer has a plurality of first absorbing holes and a plurality of second absorbing holes aligned with the first absorbing holes. The adhering layer is porous and abuts between the coverage layer and the fixed-shaped layer. The fixing tap is arranged on the coverage layer in order to fasten the minus-pressured bandage on the wound. In this arrangement, a minus-pressured suction applied to the wound may be increased to facilitate capillary angiogenesis.

### BACKGROUND OF THE INVENTION

1. Field of the Invention

The present invention relates generally to a medical apparatus, and more particularly, to a minus-pressured bandage.

2. Description of the Related Art

According to clinical researches, applying a minus pressure to a tissue compartment can reinforce and accelerate the growth of new tissues at this tissue compartment. In the medical field, it is called “minus-pressured therapy”, “antihypertensive therapy”, or “vacuum therapy”, which is applied to wound healing. Such therapy includes many advantages, such as healing of granulation tissues and angiogenesis.

Referring to FIG. 1, a conventional minus-pressured device includes a covering layer 81 and an absorbent layer 92. The covering layer 91 includes two combination portions 911 formed at two opposite lateral edges thereof separately. The two combination portions 911 can be fastened with each other. The covering layer 91 includes a conduit 912 formed at a center thereof. The absorbent layer 92 is made of a porous material, like a sponge.

Referring to FIG. 2 in view of FIG. 1, when the conventional minus-pressured device is operated, the absorbent layer 92 is laid on a user's wound first and then the covering layer 91 is put on the absorbent layer 92 and wound around the human body, like four limbs. Next, the two combination portions 911 can fasten the covering layer 91. The conduit 912 can be connected with a minus-pressured device 93 via a duct 931 to allow the minus-pressured device 92 to decompress what the covering layer 91 covers.

The conventional minus-pressured device 93 can generate and apply minus-pressured suction to the wound through the porous structure of the absorbent layer 92, such that the tissues at the wound undergo the minus-pressured suction, namely micro mechanical force, to promote capillary angiogenesis.

However, the absorbent layer 92 is made of the porous material which is flexible, so the absorbent layer 92 can be squeezed by the minus pressure to be deformed, while the minus-pressured device 93 decompresses what the covering layer 91 covers, to further deform the pores of the covering layer 91. In this way, the minus-pressured suction of the pores can be adversely affected and even the pores can be occluded, thus bringing adverse influence to the micro mechanical force applied to the tissues at the wound and applying adverse influence to the capillary angiogenesis at the wound tissues.

### SUMMARY OF THE INVENTION

The primary objective of the present invention is to provide a minus-pressured bandage, which can enhance the minus-pressured suction applied to the wound tissues to promote the capillary angiogenesis.

The secondary objective of the present invention is to provide a minus-pressured bandage, which can more perfectly fit the wound.

Another objective of the present invention is to provide a minus-pressured bandage, which can be trimmed according to the size of the wound.

The foregoing objectives of the present invention are attained by the minus-pressured bandage composed of a coverage layer, a minus-pressured acting member, an adhering layer, and a fixing tap. The minus-pressed acting member includes a fixed-shaped layer and a contact layer abutting against the fixed-shaped layer. The contact layer is made of a resilient material. The fixed-shaped layer has hardness greater than that of the contact layer. Each of the fixed-shaped layer and the contact layer has a plurality of first absorbing holes and a plurality of second absorbing holes aligned with the first absorbing holes separately. The adhering layer is porous and abuts between the coverage layer and the fixed-shaped layer. The fixing tap is mounted to the coverage layer in order to fasten the minus-pressured bandage on the wound.

### BRIEF DESCRIPTION OF THE DRAWINGS

FIG. 1 is an exploded view of the prior art.

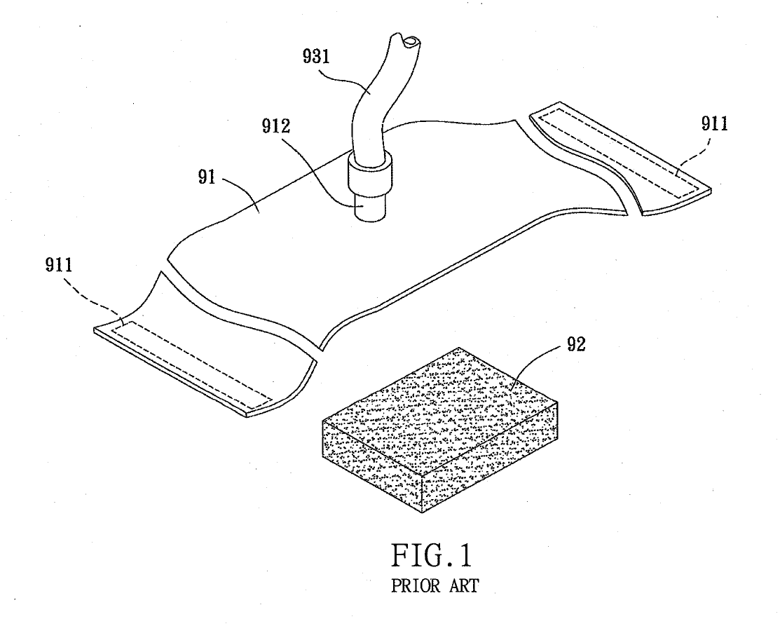

FIG. 2 is a sectional view of the prior art.

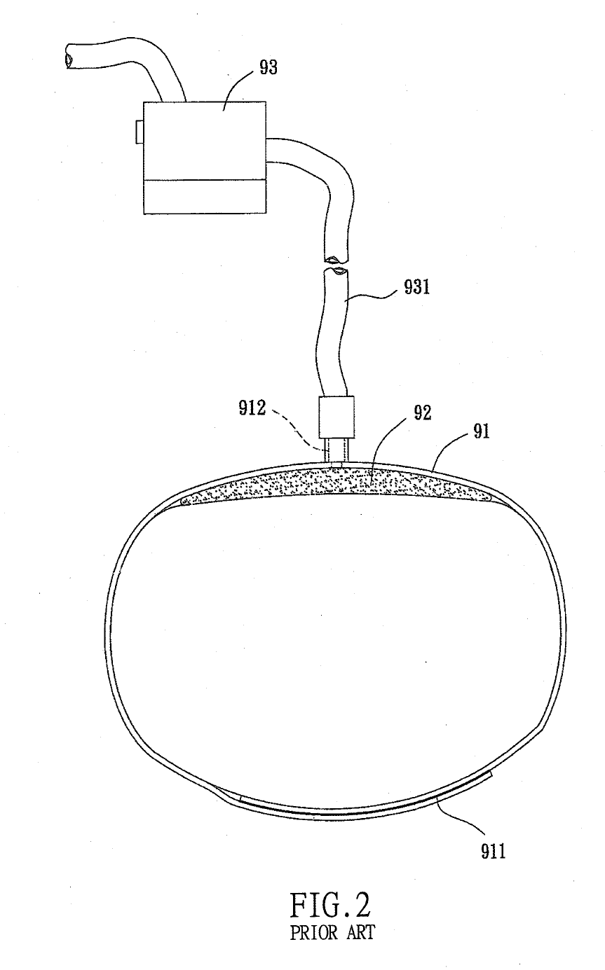

FIG. 3 is an exploded view of the first preferred embodiment of the present invention.

FIG. 4 is a sectional view of the first preferred embodiment of the present invention.

FIG. 5 is a schematic view of the first preferred embodiment of the present invention at work.

FIG. 6 is an enlarged view of a part taken from FIG. 5.

FIG. 7 is a sectional view of a second preferred embodiment of the present invention.

FIG. 8 is a schematic view of the second preferred embodiment of the present invention at work.

### DETAILED DESCRIPTION OF PREFERRED EMBODIMENTS

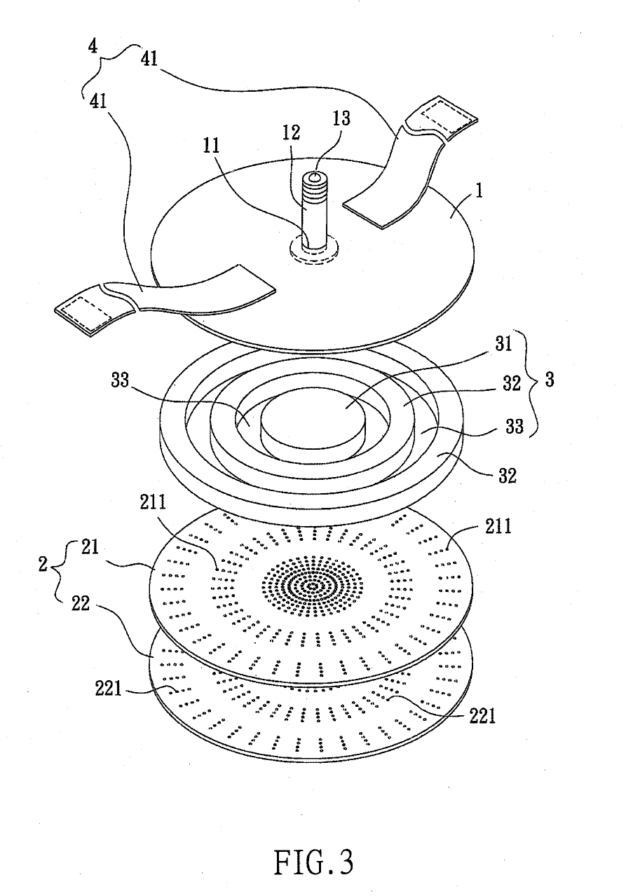

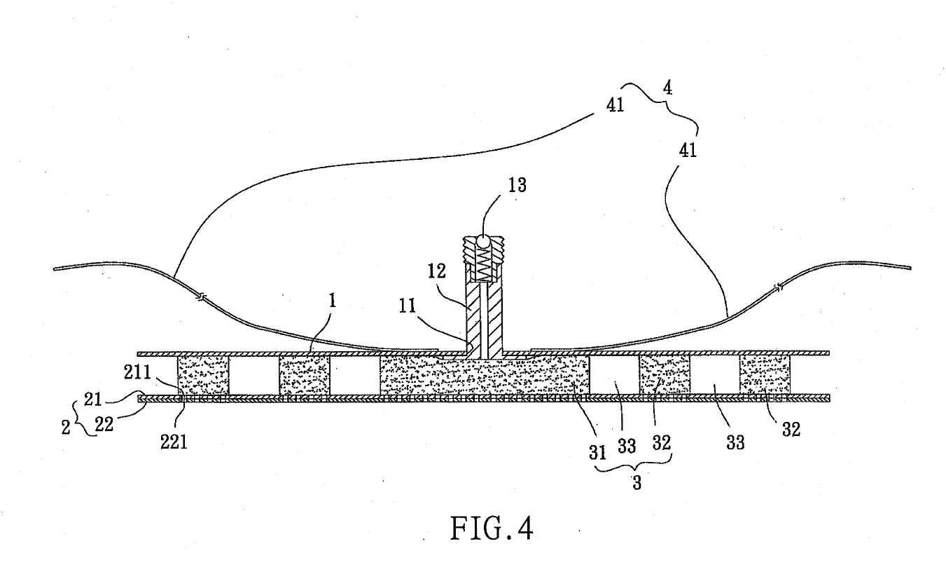

Referring to FIGS. 3-4, a minus-pressured bandage is composed of a coverage layer 1, a minus-pressured acting member 2, an adhering layer 3, and a fixing tap 4. The adhering layer 3 is mounted between the coverage layer 1 and the minus-pressured acting member 2. The minus-pressured acting member 2 can be laid on the user's wound for minus-pressured therapy. The fixing tap 4 is mounted to the coverage layer 1 for fastening the minus-pressured bandage to the wound in such a way that the minus-pressured bandage can be prevented from disengagement or displacement with respect to the wound. The detailed descriptions and operations of these elements as well as their interrelations are recited in the respective paragraphs as follows.

The coverage layer 1 is made of a deformable metal or plastic; preferably, the coverage layer 1 is made of a metallic sheet, like aluminum sheet. The coverage layer 1 includes a through hole 11. A conduit 12 is mounted to the through hole 11. The conduit 12 has a first end protruding from the coverage layer 1 in one piece or separably. The conduit 12 has a second end provided with a check valve 13.

A minus-pressured acting member 2 includes a fixed-shape layer 21 and a contact layer 22. The fixed-shaped layer 21 is made of a deformable metal or plastic; preferably, the minus-pressured acting member 2 is made of a metallic sheet, like aluminum sheet. The contact layer 22 is made of a resilient material, which is preferably PVC or a biomedical one. The fixed-shaped layer 21 has hardness greater that of the contact layer 22. The fixed-shaped layer 21 has a plurality of first absorbing holes 211. The contact layer 22 has a plurality of second absorbing holes 221. Besides, the contact layer 22 has thickness preferably greater than but at most not double as much as that of the fixed-shaped layer 21.

The adhering layer 3 abuts between the coverage layer 1 and the fixed-shaped layer 21 and is made of a porous material, which is preferably a sponge. The adhering layer 3 includes a central adhering member 31 and at least one annular adhering member 32. In this embodiment, for illustration, one central adhering member 31 and two annular adhering members 32 are taken as an example. Each of the central adhering member 31 and the annular adhering members 32 can be circular, elliptic, or rectangular. The two annular adhering members 32 have different sizes and surround the central adhering member 31. The central adhering member 31 is located with respect to the through hole 11. Besides, the relatively smaller adhering member 32 has an internal diameter greater than an external diameter of the central adhering member 31, and the relatively bigger adhering member 32 has an internal diameter larger than an external diameter of the relatively smaller adhering member 32, such that two gaps 33 are formed between one of the annular adhering members 32 and the central adhering member 31 and between the two annular adhering members 32 separately. Each of the first and second absorbing holes 211 and 221 are preferably located within the range that the central adhering member 31 abuts against the two annular adhering members 32.

The fixing tap 4 is mounted to the coverage layer 1 and is composed of two conventional Velcro® (hook and loop) straps 41 in this embodiment. Each of the Velcro® straps 41 includes a first end and a second end. The first ends of the two Velcro® straps 41 are fixed to the coverage layer 1 and preferably located over the central adhering member 31. The second ends of the Velcro® straps 41 can adhere to each other.

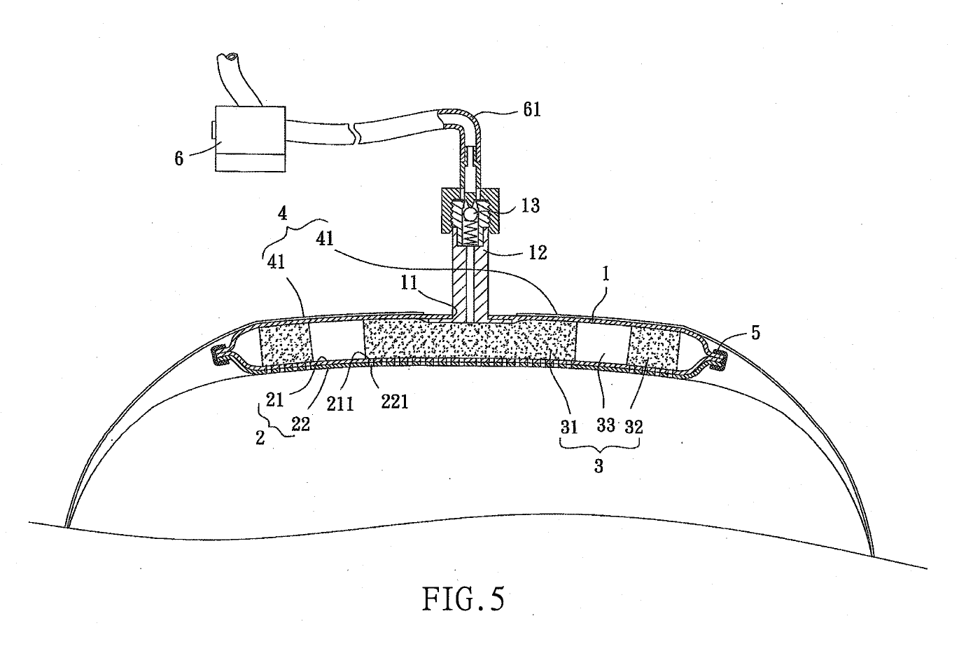

When the minus-pressured bandage is operated, external peripheral edges of the coverage layer 1 and the minus-pressured acting member 2 can be bent, abut against, and fastened to each other to form a closed space between the coverage layer 1 and the minus-pressured acting member 2. Referring to FIG. 5, when the wound that the minus-pressured therapy is to be applied to is smaller, the coverage layer 1 and the minus-pressured acting member 2 can be trimmed to a proper size along one of the gaps 33 and then the external peripheral edges of the trimmed coverage layer 1 and minus-pressured acting member 2 can be bent and abut against each other; a fastener 5 (e.g. adhesive tap) can be adhesively fastened o the external peripheral edges of the trimmed coverage layer 1 and the minus-pressured acting member 2 to also form a closed space therebetween.

When the present invention is used, the contact layer 22 is laid on the wound. Because the coverage layer 1 and the fixed-shaped layer 21 are deformable, the minus-pressured bandage can be deformed according to the wound and its ambient human curve and fixedly shaped to perfectly fit the human body. Besides, the contact layer 22 is resilient to be deformable, so it can perfectly fit the wound tissues and ambient skin thereof. Next, the two Velcro® straps 41 can be wound around the human body to fasten the minus-pressured bandage to the wound to prevent it from disengagement or displacement.

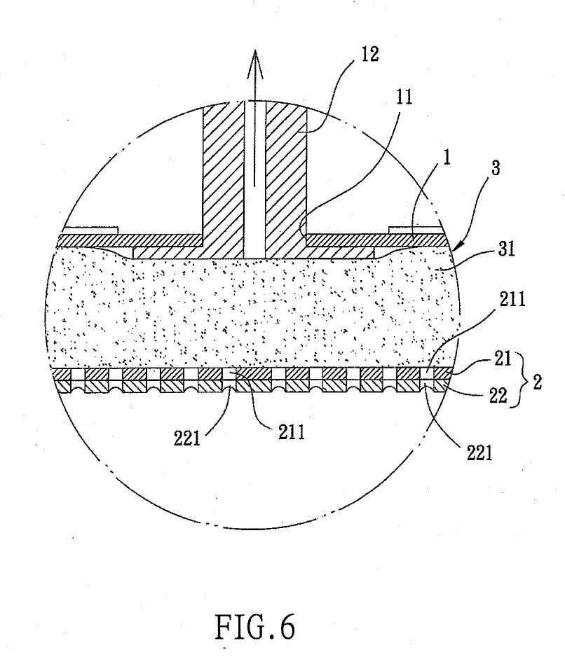

Referring to FIGS. 5-6, the conduit 12 can be connected with a minus-pressured device 6 via a duct 61 to allow the minus-pressured device 6 to decompress the closed space between the coverage layer 1 and the minus-pressured acting member 2 and to apply minus-pressured suction to the wound through the first and second absorbing holes 211 and 221 to allow the wound tissues to undergo the minus-pressured suction in the first and second absorbing holes 211 and 221. Because the contact layer 22 is connected to the fixed-shaped layer 21, it is though resilient but has sufficient fixed-shape strength to prevent the second absorbing holes 221 from deformation resulting from minus-pressured squeeze to further promote capillary angiogenesis at the wound tissues.

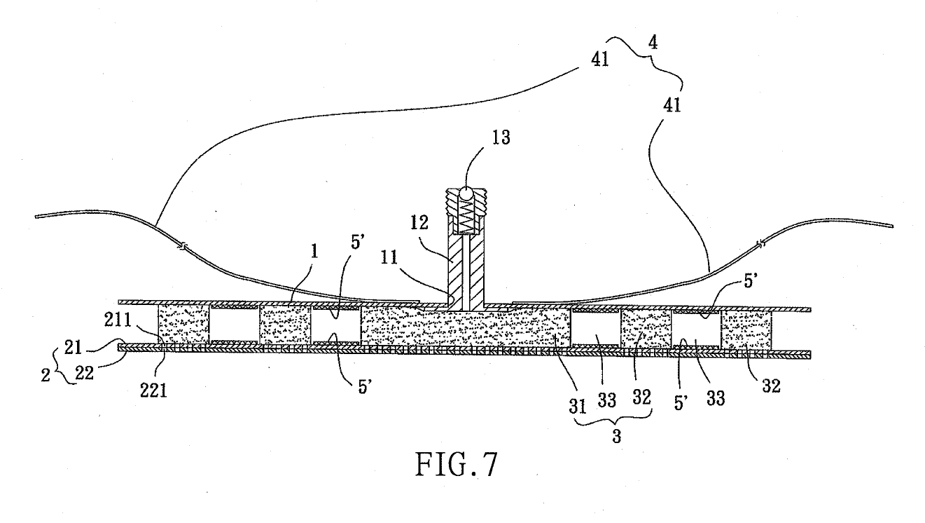

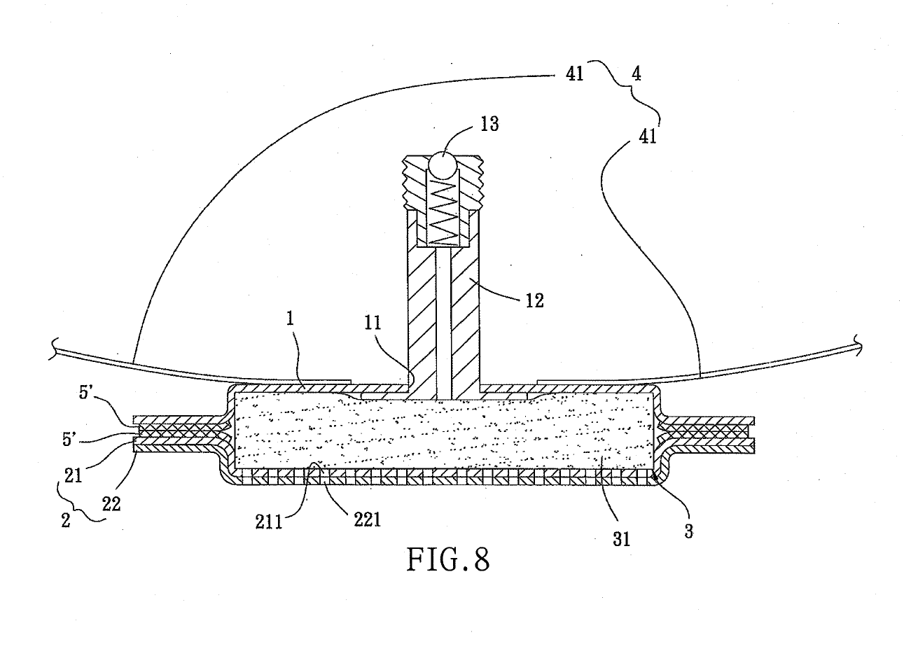

Referring FIGS. 7-8, a minus-pressured bandage constructed according to a second embodiment of the present invention is similar to that of the first embodiment, having the following difference. The minus-pressured bandage further includes a plurality of fixing members 5′ mounted to internal sides of the coverage layer 1 and the fixed-shape layer 21 separately, which face each other. Each of the fixing members 5′ is preferably mounted to the gap 33 and can adhere to the other adjacent fixing member 5′. Thus, after the coverage layer 1 and the minus-pressured acting member 2 are trimmed along one of the gaps 33 according to the size of the wound that the minus-pressured therapy is to be applied, the trimmed external peripheral edges of the coverage layer 1 and the minus-pressured acting member 2 can be adhesively fastened via the fixing members 5′.

As set above, the contact layer is connected to the fixed-shape layer. The fixed-shaped layer can be a metallic sheet to enable the contact layer to have sufficient fixed-shape strength though it is resilient to prevent the second absorbing holes from deformation resulting from the minus-pressured squeeze and to enhance the minus-pressured suction applied to the wound tissues for promotion of capillary angiogenesis.

In conclusion, the coverage layer and the fixed-shape layer of the present invention both are deformable, so the minus-pressured bandage can be deformed according to the wound and its ambient human curve. Besides, after the minus-pressured bandage is bent, it can still be fixedly shaped to keep perfectly fitting the human body. Moreover, the coverage layer and the minus-pressured acting member can trimmed to a proper size subject to the size of the wound that the minus-pressured therapy is to be applied to.

Although the present invention has been described with respect to specific preferred embodiments thereof, it is in no way limited to the specifics of the illustrated structures but changes and modifications may be made within the scope of the appended claims.

1. A minus-pressured bandage comprising:
a coverage layer having a through hole for connection with a minus-pressured device;
a minus-pressured acting member having a fixed-shape layer and a contact layer abutting against the fixed-shaped layer, the contact layer being made of a resilient material, the fixed-shaped layer having hardness greater than that of the contact layer, each of the fixed-shaped layer and the contact layer having a plurality of first absorbing holes and second absorbing holes, the first absorbing holes being aligned with the second absorbing holes respectively;
an adhering layer made of a porous material and adhering between the coverage layer and the fixed-shape layer; and
a fixing tap mounted to the coverage layer to fasten the minus-pressured bandage to the wound.

2. The minus-pressured bandage as defined in claim 1, wherein the adhering layer comprises a central adhering member and an annular adhering member, the central adhering member being located with respect to the through hole, the annular adhering member surrounding the central adhering member, a gap being formed between the central adhering member and the annular adhering member.

3. The minus-pressured bandage as defined in claim 2, wherein the adhering layer comprises a plurality of annular adhering members surrounding the annular adhering member, each two annular adhering members having a gap therebetween.

4. The minus-pressured bandage as defined in claim 3, wherein the first absorbing holes are located within the ranges that the central adhering member and the annular adhering layers abutting against the minus-pressured acting member respectively.

5. The minus-pressured bandage as defined in claim 3, wherein each of the internal sides of the coverage layer and fixed-shaped layer in the gaps comprises a fixing member.

6. The minus-pressured bandage as defined in claim 1, wherein the coverage layer and the minus-pressured acting member comprise external peripheral edges which can be hermetically connected with each other.

7. The minus-pressured bandage as defined in claim 2, wherein the fixing tap comprises two hook and loop straps, each of which has a first end and a second end, the first ends of the two hook and loop straps being fixed to the coverage layer, the second ends of the two hook and loop straps being able to adhere to each other.

8. The minus-pressured bandage as defined in claim 1, wherein each of the coverage layer and the fixed-shaped layer is made of a deformable metallic or plastic material.

9. The minus-pressured bandage as defined in claim 8, wherein each of the coverage layer and the fixed-shaped is made of aluminum.

10. The minus-pressured bandage as defined in claim 1, wherein the contact layer is made of PVC.
